SEQUENCE LISTING (10 sequences)

SEQ ID NO: 1 - DNA, 69 bp, synthetic construct
  source 1..69: mol_type = other DNA; note = A
tttttcaacatcagtctgataagctagcctttaatacgtagcttatcagatttttttaccaagcttggt

SEQ ID NO: 2 - DNA, 67 bp, synthetic construct
  source 1..67: mol_type = other DNA; note = B
ttttgataagctacgtattaaaggcatgatgaactgagcctttaatacgttttttaccaagcttggt

SEQ ID NO: 3 - DNA, 67 bp, synthetic construct
  source 1..67: mol_type = other DNA; note = C
tttattaaaggctcagttcatcagtagcttatcagactgatcaactgagctttttaccaagcttggt

SEQ ID NO: 4 - RNA, 22 nt, synthetic construct
  source 1..22: mol_type = other RNA; note = miRNA-21
tagcttatcagactgatgttga

SEQ ID NO: 5 - RNA, 21 nt, synthetic construct
  source 1..21: mol_type = other RNA; note = miRNA-27
ttcacagtggctaagttccgc

SEQ ID NO: 6 - RNA, 24 nt, synthetic construct
  source 1..24: mol_type = other RNA; note = miRNA-155
ttaatgctaatcgtgataggggtt

SEQ ID NO: 7 - RNA, 23 nt, synthetic construct
  source 1..23: mol_type = other RNA; note = miRNA-373
gaagtgcttcgattttggggtgt

SEQ ID NO: 8 - RNA, 22 nt, synthetic construct
  source 1..22: mol_type = other RNA; note = SM miRNA-21
tagcttatcagactgatgttca

SEQ ID NO: 9 - RNA, 22 nt, synthetic construct
  source 1..22: mol_type = other RNA; note = DM miRNA-21
tagcttatcagactgatattca

SEQ ID NO: 10 - RNA, 22 nt, synthetic construct
  source 1..22: mol_type = other RNA; note = TM miRNA-21
tagcttatcagattgatattca

(19) United States
(12) Patent Application Publication
(10) Pub. No.: US 2025/0075262 A1
(43) Pub. Date: Mar. 6, 2025

(54) METHOD, SYSTEM, AND KIT FOR IN SITU DETECTION OF MICRO RIBONUCLEIC ACID (miRNA) CARRIED BY EXTRACELLULAR VESICLES (EVs)

(71) Applicant: Qilu University of Technology (Shandong Academy of Sciences), Jinan (CN)

(72) Inventors: Yingshu Guo, Jinan (CN); Di Wu, Jinan (CN)

(21) Appl. No.: 18/250,496

(22) PCT Filed: Nov. 25, 2022
(86) PCT No.: PCT/CN2022/134415

§ 371 Date: Apr. 25, 2023

(30) Foreign Application Priority Data
Sep. 29, 2022 (CN) 202211197375.2

(51) Int. Cl. C12Q 1/6841

(52) U.S. Cl. CPC C12Q 1/6841

(57) ABSTRACT

The present disclosure belongs to the technical field of detection, and specifically relates to a method, a system, and a kit for in situ detection of a micro ribonucleic acid (miRNA) carried by extracellular vesicles (EVs). In the method for in situ detection of a miRNA carried by EVs, fluorescence detection probes are delivered without disrupting a membrane vesicle structure of the EVs using an innovative strategy of conducting membrane fusion between red blood cell membrane (RBCM)-derived vesicles (RVs) and the EVs. Therefore, an obvious increase in a local concentration of the fluorescence probes in a confined space leads to a sharp increase in a probability of collisions between the probes, thereby increasing a detection sensitivity of a target. There is a desirable linear relationship between a detected fluorescence intensity and a miRNA-21 concentration at 50 pM to 40 nM.

### CROSS REFERENCE TO RELATED APPLICATION

The present application is a U.S. National Phase application of PCT International Application Number PCT/CN2022/134415, filed on Nov. 25, 2022, which claims priority to the Chinese Patent Application No. 202211197375.2, filed with the China National Intellectual Property Administration (CNIPA) on Sep. 29, 2022, and entitled “METHOD, SYSTEM, AND KIT FOR IN SITU DETECTION OF MICRO RIBONUCLEIC ACID (miRNA) CARRIED BY EXTRACELLULAR VESICLES (EVs)”, which is incorporated herein by reference in its entirety.

### REFERENCE TO SEQUENCE LISTING

A computer readable XML file entitled “BGI021_002APC”, that was created on Apr. 24, 2023, with a file size of about 11,828 bytes, contains the sequence listing for this application, has been filed with this application, and is hereby incorporated by reference in its entirety.

### TECHNICAL FIELD

The present disclosure belongs to the technical field of detection, and specifically relates to a method, a system, and a kit for in situ detection of a micro ribonucleic acid (miRNA) carried by extracellular vesicles (EVs).

### BACKGROUND

MicroRNAs (miRNAs) are single-stranded, short (including approximately 19 to 23 nucleotides), endogenous, and non-coding regulatory RNAs that are critical in many biological processes. As an important link in the gene expression, miRNAs have been identified as an extremely promising biomarker. Since their abnormal expression levels are closely related to many diseases including cancer, the miRNAs have a strong predictive value in the diagnosis and prognosis of cancer. For example, miRNA-21 was validated as a potential novel oncogene that regulates tumor cell cycle and tumor metastasis. However, miRNAs have some properties, such as small size, high sequence similarity, easy degradation, and especially low abundance. Therefore, it is a highly challenging task in high-sensitivity detection of low-level miRNAs.

### SUMMARY

In order to solve the above problems, the present disclosure provides a method, a system, and a kit for in situ detection of a miRNA carried by EVs. In the present disclosure, a fluorescence detection probe is delivered without destroying a membrane vesicle structure of the EVs, so as to realize in-situ fluorescence detection of the miRNA in a confined space, showing a high sensitivity.

The present disclosure provides a method for in situ detection of a miRNA carried by EVs, including the following steps: subjecting red blood cell membrane (RBCM)-derived vesicles (RVs) to membrane fusion with EVs, and delivering a specific hairpin probe designed for a target miRNA into the EVs to complete DNA self-assembly, thereby achieving in situ fluorescence detection of the miRNA carried by the EVs.

Preferably, there are three specific hairpin probes; sequences of the three specific hairpin probes each include a self-complementary sequence and a complementary palindromic sequence; and two of the specific hairpin probes each are modified with a fluorophore and a quencher.

Preferably, the target miRNA includes miRNA-21.

Preferably, a specific hairpin probe designed for the miRNA-21 includes A-Cy5, B, and C-Cy5; A has a nucleotide sequence shown in SEQ ID NO: 1, and the sequence shown in SEQ ID NO: 1 has a quencher modified on T at a 11th position and a fluorophore modified on T at a 53rd position from a 5′-end to a 3′-end;

B has a nucleotide sequence shown in SEQ ID NO: 2; and

C has a nucleotide sequence shown in SEQ ID NO: 3, and the sequence shown in SEQ ID NO: 3 has a fluorophore modified on T at a 6th position and a quencher modified on T at a 54rd position from a 5′-end to a 3′-end.

Preferably, the fluorophore includes Cy5, and the quencher includes BHQ2.

The present disclosure further provides a system for in situ detection of a miRNA-21 carried by EVs, including a miRNA-21 standard and a specific hairpin probe designed for the miRNA-21, where the specific hairpin probe includes A-Cy5, B, and C-Cy5; A has a nucleotide sequence shown in SEQ ID NO: 1, and the sequence shown in SEQ ID NO: 1 has BHQ2 modified on T at a 11th position and Cy5 modified on T at a 53rd position from a 5′-end to a 3′-end;

B has a nucleotide sequence shown in SEQ ID NO: 2; and

C has a nucleotide sequence shown in SEQ ID NO: 3, and the sequence shown in SEQ ID NO: 3 has Cy5 modified on T at a 6th position and BHQ2 modified on T at a 54rd position from a 5′-end to a 3′-end.

The present disclosure further provides a kit for in situ detection of a miRNA-21 carried by EVs, including the system, an RVs extraction reagent, and an EVs extraction reagent.

The present disclosure further provides a method for in situ detection of a miRNA-21 carried by EVs based on the system or the kit, including the following steps: mixing the A-Cy5, the B, and the C-Cy5 that have a same final concentration with the miRNA-21 standards with different concentrations, conducting incubation at 37° C. for 120 min, conducting spectrofluorimetry and drawing a standard curve;
extracting the RVs with the RVs extraction reagent, mixing a resulting RBCM suspension with the A-Cy5, the B, and the C-Cy5, and conducting co-extrusion to obtain RVs; and
mixing the RVs with the EVs extracted by the RVs extraction reagent, conducting incubation at 37° C. for 120 min, and conducting the same spectrofluorimetry.

Preferably, the A-Cy5, the B, and the C-Cy5 each have a final concentration of 200 nM.

Preferably, the RVs have a particle size of 122 nm to 396 nm.

Beneficial effects: in the method for in situ detection of a miRNA carried by EVs, fluorescence detection probes are delivered without disrupting a membrane vesicle structure of the EVs using an innovative strategy of conducting membrane fusion between the RVs and EVs. Therefore, an obvious increase in a local concentration of the fluorescence probes in a confined space leads to a sharp increase in a probability of collisions between the probes, thereby increasing a detection sensitivity of a target. The present disclosure further designs a fluorescent probe with a high sensitivity and an excellent selectivity for the miRNA-21, where DNA self-assembly is conducted to form a DNA nanosphere structure to realize the fluorescence detection. As verified by the examples, there is a desirable linear relationship between a detected fluorescence intensity and a miRNA-21 concentration at 50 pM to 40 nM. Based on the above membrane fusion strategy, the probe is delivered into EVs to form a nanoscale confined space, and DNA self-assembly occurs in the space, realizing the in situ fluorescence detection of miRNA-21 in EVs.

### BRIEF DESCRIPTION OF THE DRAWINGS

To describe the embodiments of the present disclosure or the technical solutions in the related art more clearly, the accompanying drawings required in the embodiments are briefly introduced below. Obviously, the accompanying drawings described below are only some embodiments of the present disclosure. A person of ordinary skill in the art may further obtain other accompanying drawings based on these accompanying drawings without creative labor.

FIG. 1 shows a schematic diagram for a principle of in situ detection of miRNA-21 by DNA self-assembly;

FIG. 2 shows a transmission electron microscopy (TEM) image (left) and a particle size distribution map (right) of DNA NS;

FIG. 3 shows a polyacrylamide gel electrophoretogram of a reaction product of the DNA self-assembly, where lanes represent in sequence from left to right as follows: single-stranded miRNA-21; reaction of miRNA-21 and probe A; reaction of miRNA-21, probe B, and probe C; reaction of miRNA-21, probe A, probe B, and probe C; single-stranded probe A; single-stranded probe B; and single-stranded probe C;

FIG. 4 shows a schematic diagram of a principle of in situ detection of the miRNA-21 in EVs using an RV strategy;

FIG. 5 shows a TEM image (left) and a particle size distribution map (right) of EVs;

FIG. 6 shows a TEM image (left) and a particle size distribution map (right) of RVs;

FIG. 7 shows potential images of EVs, RVs, and incubation of both;

FIG. 8 shows a standard regression line of an absorbance of different concentrations of protein solutions at 562 nm;

FIG. 9 shows feasibility of using a DNA self-assembly system for miRNA-21 detection proved by fluorescence measurement;

FIG. 10 shows a fluorescence spectrum when there are different concentrations of miRNA-21;

FIG. 11 shows a linear relationship (left) and a standard regression line (right) between a fluorescence intensity and a concentration of the target miRNA-21;

FIG. 12 shows fluorescence intensities detected by the RV strategy for 7 different miRNAs;

FIG. 13 shows a detection result of a stability of the DNA self-assembly within 8 h; and

FIG. 14 shows kinetic curves of the miRNA-21 detected by fluorescent probes in a solution (red) and in EVs (black).

### DETAILED DESCRIPTION OF THE EMBODIMENTS

The present disclosure provides a method for in situ detection of a miRNA carried by EVs, including the following steps: subjecting RVs to membrane fusion with EVs, and delivering a specific hairpin probe designed for a target miRNA into the EVs to complete DNA self-assembly, thereby achieving in situ fluorescence detection of the miRNA carried by the EVs.

In the present disclosure, since there are no complicated organelles inside the RBCs, the difficulty of extracting RBCMs can be reduced. A sialic acid is present on a surface of the RBCM, and a sialic acid receptor is present on a membrane surface of the EV. The specific combination of these two membrane proteins makes a connection between RVs and EVs, and promotes the membrane fusion between the RVs and EVs. Based on the membrane fusion of RVs and EVs, internal molecules of the EVs can be fixed in a confined space without destroying the membrane vesicle structure of EVs, which significantly increases a probability of collision between reactants, thus improving the reaction efficiency.

In the present disclosure, there are preferably three specific hairpin probes; sequences of the three specific hairpin probes each include a self-complementary sequence and a complementary palindromic sequence; and two of the specific hairpin probes each are modified with a fluorophore and a quencher. There is no special limitation on a type of the target miRNA; in the example, miRNA-21 is taken as an example for illustration, but cannot only be regarded as an entire protection scope of the present disclosure.

In the present disclosure, a specific hairpin probe designed for miRNA-21 includes preferably A-Cy5, B, and C-Cy5, and their sequence information is shown in Table 1. In the sequences shown in Table 1, the underlined fragments represent the self-complementary stem portion of the hairpin probe; the segments in italics are complementary palindromic sequences; and the bold black fragments are the modified fluorophore and quencher on the probe. There is no special limitation on selection of the fluorophore and quencher; in the example, BHQ2 is used as the quencher, and Cy5 is used as the fluorophore, but they cannot only be regarded as an entire protection scope of the present disclosure.

TABLE 1
Sequence information involved in the present disclosure

| Name | Sequence (5′-3′) | SEQ ID NO |
|---|---|---|
| miRNA-21 | UAG CUU AUC AGA CUG AUG UUG A | 4 |
| A | TT TTT CAA CAT CAG TCT GAT AAG CTA GCC TTT AAT ACG TAG CTT ATC AGA | 1 |
| | TTT TTT T ACCAAG CTTGGT | |
| B | TTTT GAT AAG CTA CGT ATT AAA GGC ATG ATG AAC TGA GCC TTT AAT ACG | 2 |
| | TTT TTT ACCAAG CTTGGT | |
| C | TTT ATT AAA GGC TCA GTT CAT CAG TAG CTT ATC AGA CTG ATC AAC TGA | 3 |
| | GCT TTT T ACCAAG CTTGGT | |
| A-Cy5 | 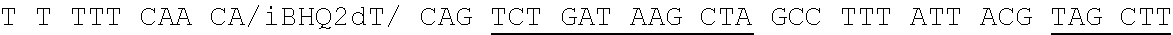 | |
| | 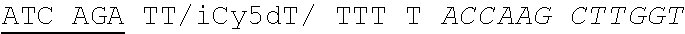 | |
| C-Cy5 | 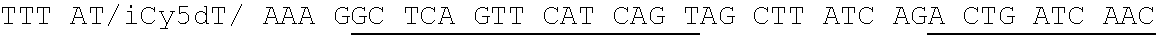 | |
| | 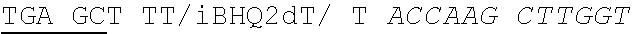 | |
| miRNA-27 | UUC ACA GUG GCU AAG UUC CGC | 5 |
| miRNA-155 | UUA AUG CUA AUC GUG AUA GGG GUU | 6 |
| miRNA-373 | GAA GUG CUU CGA UUU UGG GGU GU | 7 |
| SM miRNA-21 | UAG CUU AUC AGA CUG AUG UUC A | 8 |
| DM miRNA-21 | UAG CUU AUC AGA CUG AUA UUC A | 9 |
| TM miRNA-21 | UAG CUU AUC AGA UUG AUA UUC A | 10 |

The present disclosure further provides a system for in situ detection of a miRNA-21 carried by EVs, including a miRNA-21 standard and a specific hairpin probe designed for the miRNA-21, where the specific hairpin probe includes A-Cy5, B, and C-Cy5; A has a nucleotide sequence shown in SEQ ID NO: 1, and the sequence shown in SEQ ID NO: 1 has BHQ2 modified on T at a 11th position and Cy5 modified on T at a 53rd position from a 5′-end to a 3′-end;
B has a nucleotide sequence shown in SEQ ID NO: 2; and
C has a nucleotide sequence shown in SEQ ID NO: 3, and the sequence shown in SEQ ID NO: 3 has Cy5 modified on T at a 6th position and BHQ2 modified on T at a 54rd position from a 5′-end to a 3′-end.

In the present disclosure, the miRNA-21 standard product has a sequence preferably shown in SEQ ID NO: 4, and the miRNA-21 standard product needs to be diluted to different working concentrations with a PBS to draw a standard curve. For example, in the example, the miRNA-21 standard has the working concentration set to 0, 10 pM, 50 pM, 100 pM, 500 pM, 1 nM, 5 nM, 10 nM, 20 nM, 30 nM, 40 nM, 50 nM, 100 nM, 150 nM, and 200 nM.

The present disclosure further provides a kit for in situ detection of a miRNA-21 carried by EVs, including the system, an RV extraction reagent, and an EVs extraction reagent.

In the present disclosure, the RV extraction reagent includes preferably a reagent required for preparation of the RVs with a particle size of about 200 nm by extrusion. The EVs extraction reagent is preferably a rapid exosome extraction kit common in the art. For example, in the example, the kit is purchased from Yeasen Biotechnology (Shanghai) Co., Ltd. During extraction:

5 times the volume of deionized water is added to a purchased 2% mouse RBC suspension, mixed well, and allowed to stand at 4° C. for 1 h to make the RBCs absorb water and burst. The hemoglobin in the suspension is removed by centrifugation at 14,000 R for 10 minutes, a supernatant is discarded, and a bottom pellet is washed three times with PBS. An obtained pale pink RBCM precipitate is dispersed with 1 mL of a PBS solution; a resulting dispersion is repeatedly extruded 5 times and 10 times with a 0.45 μm filter and a 0.22 μm filter, respectively, to finally obtain RVs with a particle size of about 200 nm. In addition, the RVs wrapping hairpin probes are prepared by co-extrusion, including: mixing the hairpin probes with an RBCM suspension while extruding repeatedly with a filter, and removing free probes in an obtained solution after extrusion with an ultrafiltration tube.

The present disclosure further provides a method for in situ detection of a miRNA-21 carried by EVs based on the system or the kit, including the following steps: mixing the A-Cy5, the B, and the C-Cy5 that have a same final concentration with the miRNA-21 standards with different concentrations, conducting incubation at 37° C. for 120 min, conducting spectrofluorimetry and drawing a standard curve;

Extracting the RVs with the RVs extraction reagent, mixing a resulting RBCM suspension with the A-Cy5, the B, and the C-Cy5, and conducting co-extrusion to obtain RVs wrapping detection probes;

Mixing the RVs with the EVs extracted by the RV extraction reagent, conducting incubation at 37° C. for 120 min, and conducting the same spectrofluorimetry.

In the present disclosure, the A-Cy5, B, and C-Cy5 each have a final concentration of preferably 200 nM. Moreover, before the spectrofluorimetry, the A-Cy5, B, and C-Cy5 at the same final concentration above, as well as the target miRNA-21 at different given concentrations are preferably added into a PCR tube and diluted to 100 μL, incubated at 37° C. for 120 min, followed by conducting the spectrofluorimetry. Preferably, the spectrofluorimetry is conducted using an F-4600 fluorescence spectrophotometer with a xenon lamp as an excitation source at an excitation wavelength of 635 nm, to record a fluorescence spectrum from 650 nm to 750 nm.

By the detection method of the present disclosure, there is a desirable linear relationship between a fluorescence intensity and a miRNA-21 concentration at 50 pM to 40 nM, and a linear equation is y=11.94x+88.895; where x represents the concentration of miRNA-21, y is a fluorescence intensity change value ΔI_{FL}. The ΔI_{FL} refers to a fluorescence intensity value at a certain moment minus a fluorescence intensity value at an initial moment, and ΔI_{FL}=I_{FLt}−I_{FL0}, demonstrating the high sensitivity of the DNA self-assembly system. When the linear equation is used to detect the concentration of miRNA-21 in the sample, the concentration of miRNA-21 in the sample can be quantified only by substituting the measured fluorescence change value.

In order to further illustrate the present disclosure, the method, the system, and the kit for in situ detection of a miRNA carried by EVs provided by the present disclosure will be described in detail below in conjunction with accompanying drawings and examples, but they should not be construed as limiting the protection scope of the present disclosure.

In the example of the present disclosure, the reagents used are common commercially-available reagents in the field unless otherwise specified. For example, all the oligonucleotides used (Table 1) are synthesized by Sangon Biotech (Shanghai) Co., Ltd., and then purified by HPLC. All DNA sequences are dissolved and diluted with PBS to a final concentration of 10 μM and stored at 4° C. until use. All MCF-7 breast cancer cells involved in the experiment are from Wuhan Procell Life Science&Technology Co., Ltd. All other reagents are of analytical grade and used directly without further purification. All water used in this experiment is sterilized ultrapure water.

In the example of the present disclosure, the morphology of nanomaterials is characterized by a SEM (JEM-2100, JEOL). The particle size and Zeta potential of the material are measured using a Zeta-Size Nano instrument (Zen 3600, Malvern Instruments Ltd.). An absorbance measurement of a BCA protein quantification experiment is realized by an American BioTek Epoch full-wavelength microplate reader. The fluorescence data in the experiment are detected and recorded by an F-4600 fluorescence spectrophotometer (Hitachi).

In order to further illustrate the present disclosure, the method, the system, and the kit for in situ detection of a miRNA carried by EVs provided by the present disclosure are described in detail below in connection with examples, but these examples should not be understood as limiting the claimed scope of the present disclosure.

### Example 1

### I. Experimental Operations

### 1.1 Synthesis of DNA Nanospheres

According to a probe concentration required for the experiment, the required miRNA and three hairpin probe stock solutions were added to a PCR tube in sequence, and diluted to 100 μL with a PBS. After reacting at 37° C. for 120 min, a resulting reaction product was stored at 4° C. for subsequent analysis.

### 1.2 Cell Culture Experiment

A medium used for MCF-7 cells in this experiment was a DMEM containing 10% fetal bovine serum and 1% diabody (penicillin-streptomycin). During cell culture, the temperature in the cell incubator was kept at 37° C. and the CO₂ concentration was 5%.

### 1.3 Polyacrylamide Gel Electrophoresis

The DNA reaction samples were subjected to 10% polyacrylamide gel electrophoresis (PAGE) at a fixed potential of 90 V, using a 1×TAE buffer as an electrophoresis buffer. In the sample pretreatment stage, the DNA solution and loading buffer had a ratio of 5:1. After electrophoresis, an electrophoresis gel was stained with a Super Red dye for 1 h, and then imaged on a gel imager.

### 1.4 Extraction of EVs

EVs were extracted and isolated from a supernatant of MCF-7 cell culture by a cell culture supernatant exosome rapid extraction kit (Yeasen Biotechnology (Shanghai) Co., Ltd.). Before extracting EVs, a normal fetal bovine serum in the cell culture medium was replaced with an exosome-free fetal bovine serum, and the extraction was conducted after three consecutive passages.

### 1.5 Preparation of RVs

RVs with a particle size of about 200 nm were prepared by extrusion. 5 times the volume of deionized water was added to a purchased 2% mouse RBC suspension, mixed well, and allowed to stand at 4° C. for 1 h to make the RBCs absorb water and burst. The hemoglobin in the suspension was removed by centrifugation at 14,000 R for 10 minutes, a supernatant was discarded, and a bottom pellet was washed three times with PBS. An obtained pale pink RBCM precipitate was dispersed with 1 mL of a PBS solution. The resulting dispersion was repeatedly extruded 5 times and 10 times with a 0.45 μm filter and a 0.22 μm filter, respectively, to finally obtain RVs with a particle size of about 200 nm. In addition, the RVs wrapping hairpin probes were prepared by co-extrusion, including: the hairpin probes were mixed with an RBCM suspension while extruding repeatedly with a filter, and free probes were removed in an obtained solution after extrusion with an ultrafiltration tube.

### 1.6 Fluorescence Measurement

Fluorescence measurement was conducted on the DNA self-assembly samples to investigate the feasibility and specificity of DNA nanosphere-based miRNA-21 detection. The synthesis of all DNA samples was conducted according to the steps mentioned above, and each sample to be tested was diluted to 100 μL with PBS for subsequent detection. The spectrofluorimetry was conducted using an F-4600 fluorescence spectrophotometer with a xenon lamp as an excitation source at an excitation wavelength of 635 nm, to record a fluorescence spectrum from 650 nm to 750 nm. Before the fluorescence measurement, the same concentration of A-Cy5, B, and C-Cy5, as well as different given concentrations of target miRNA-21 were added to a PCR tube and diluted to 100 μL. After incubation at 37° C. for 120 min, spectrofluorimetry was conducted on the samples separately. The A-Cy5, B, and C-Cy5 each had a final concentration of 200 nM; and the target miRNA-21 had final concentrations of 0, 10 pM, 50 pM, 100 pM, 500 pM, 1 nM, 5 nM, 10 nM, 20 nM, 30 nM, 40 nM, 50 nM, 100 nM, 150 nM, and 200 nM in sequence.

### II. Results and Analysis

2.1 Detection of miRNA-21 by DNA Self-Assembly Reaction

### 2.1.1 Signal Amplification Mechanism and Particle Size Determination of DNA Nanospheres

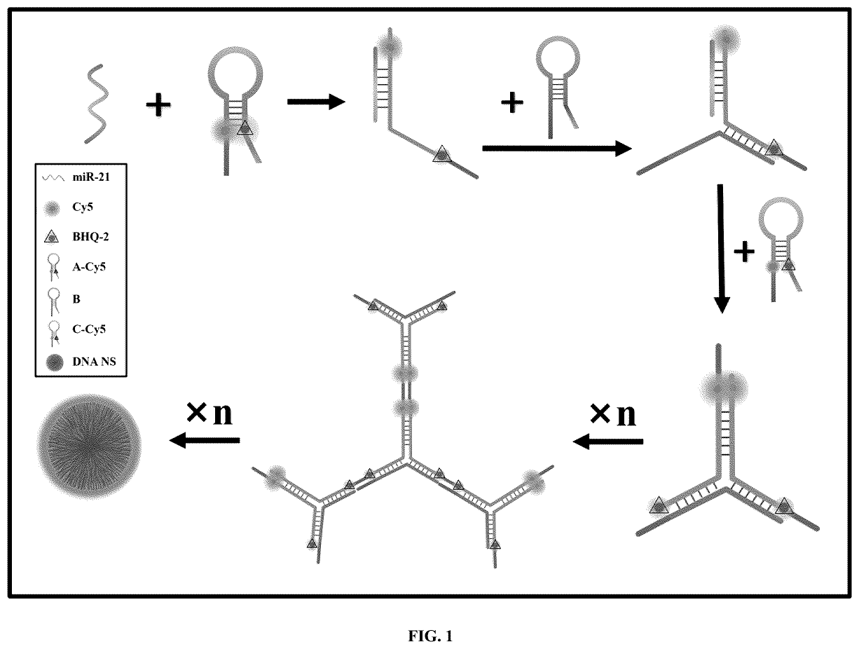

In the present disclosure, three DNA hairpin probes A, B and C were designed in Table 1, and the sequences of the three hairpin probes were complementary to each other in order to form a Y-shaped DNA structural unit. In order to observe the self-assembly of DNA to achieve fluorescence detection, a Cy5 fluorophore and a BQH-2 quencher were modified on probe A and probe C, respectively, to obtain fluorescent probes A-Cy5 and C-Cy5. In addition, a palindromic complementary sequence was added to a 3′-end of each hairpin probe, allowing the probes to be ligated end-to-end. These Y-shaped DNA structural units could also be ligated to each other through this complementary sequence to form a larger DNA structure, and these ligations were not fixed on a same plane but in an assembly process in a three-dimensional space, finally forming three-dimensional DNA nanospheres. As shown in FIG. 1, miRNA-21 and the three probes were incubated at 37° C. to allow a reaction; the target detection substance miRNA-21 could only open probe A but not probe B and probe C, and the next DNA self-assembly reaction could occur only after probe A was opened. Specifically, miRNA-21 opened the hairpin probe A-Cy5 to form a 21-A-Cy5 structure. A-Cy5 hybridized to probe B, resulting in a 21-A-Cy5-B triplex structure with more cohesive ends. C-Cy5 continued to be opened and hybridized with the 21-A-Cy5-B to form a four-stranded structure of 21-A-Cy5-B-C-Cy5. However, this RNA strand might be replaced and released under the action of C-Cy5, so as to re-participate in the next assembly reaction. In this way, a Y-shaped structural unit of A-Cy5-B-C-Cy5 was formed. Finally, a large number of the Y-shaped structural units were cross-linked through the complementarity of cohesive ends to assemble into the final DNA NS product. During the entire reaction, the opening of the A-Cy5 probe might emit a fluorescent signal, while the opening of the C-Cy5 might enhance the fluorescent signal and achieve signal amplification.

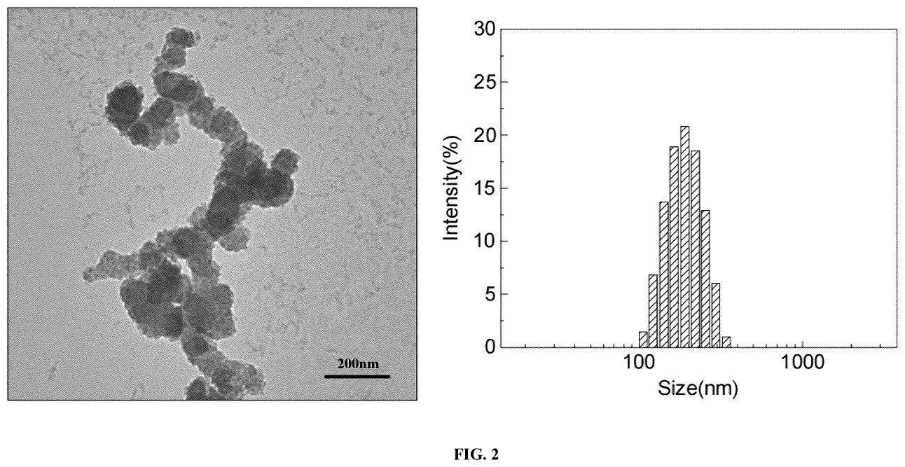

The TEM image (left image in FIG. 2) of the prepared DNA NS was taken, and the obtained image could show that the synthesis method using DNA self-assembly successfully synthesized the DNA NS. The image showed that the DNA NS had a particle size of about 200 nm and was in a spherical state.

The particle size of DNA NS was measured, and the particle size distribution map (right figure in FIG. 2) showed that the DNA NS had a particle size of about 194.5 nm, which was similar to the results of TEM, confirming that the synthesized DNA NS had a size of about 200 nm.

### 2.1.2 Verification of Feasibility of the DNA Self-Assembly

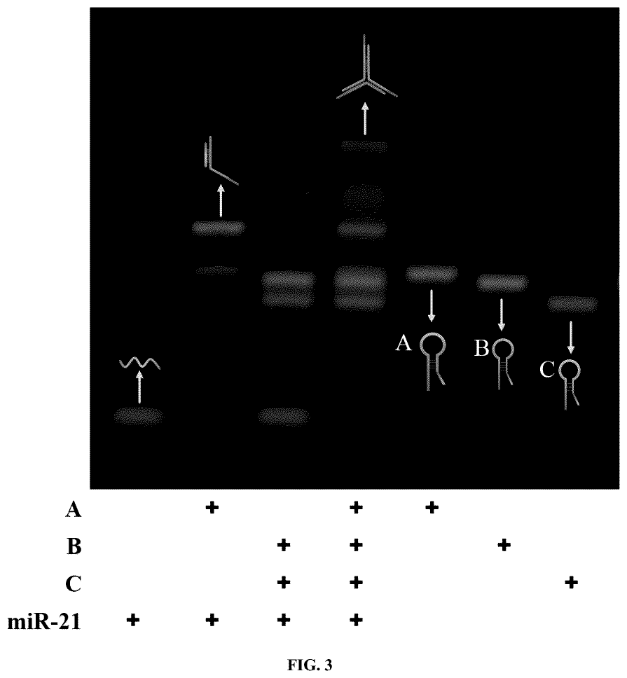

In order to verify the feasibility of the DNA self-assembly, 7 different oligonucleotide chains (Table 1) were also designed for reaction, and the 7 products were verified by polyacrylamide gel electrophoresis. The electrophoresis results were shown in FIG. 3, where the third lane from left to right, and from top to bottom corresponded to probe B, probe C, and miRNA-21, respectively, indicating that in the absence of probe A, hairpin probes B and C could not be opened; the fourth lane was a result of co-incubation reaction on the probes A, B, and C with the miRNA-21, and the top band represented the success of the self-assembly of these four nucleotide chains.

2.2 Detection of miRNA-21 in EVs by RV Strategy

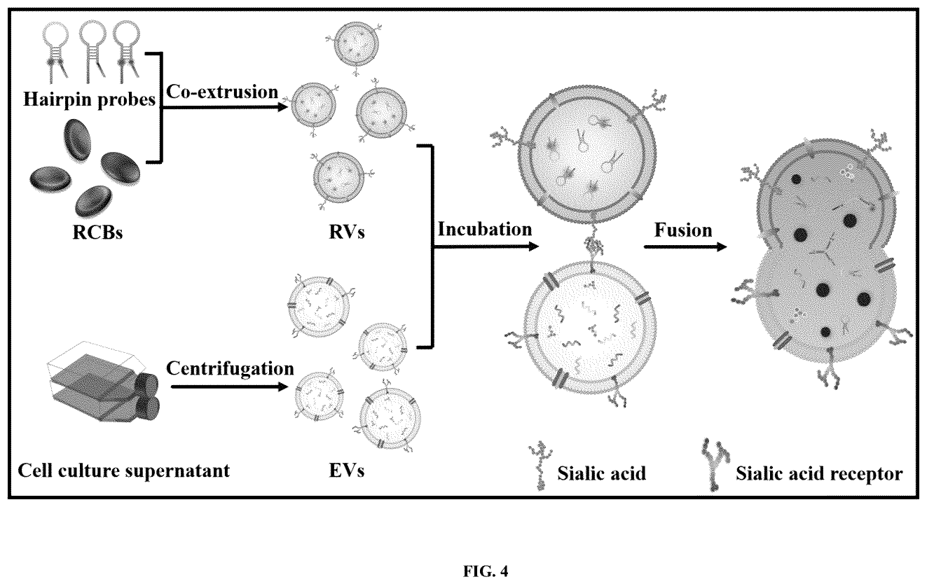

As shown in FIG. 4, the RBCM and the three designed hairpin probes were subjected to co-extrusion to prepare RVs with a uniform particle size and wrapping fluorescence detection probes, and the RVs were mixed with MCF-7 cell-derived EVs for incubation. Under the ligation between sialic acid and sialic acid receptors, RVs and EVs might come into contact, and then phospholipid bilayers of the two might fuse with each other, to finally form a larger membrane vesicle. The fluorescent probes in RVs and the biomolecules in EVs could move in this larger vesicle to undergo DNA self-assembly, and the designed hairpin probe might emit fluorescence after being opened (FIG. 1 showed a specific reaction mechanism). Finally, in situ fluorescence detection of miRNA-21 was realized inside EVs.

### 2.3 Synthesis and Characterization of EVs and RVs

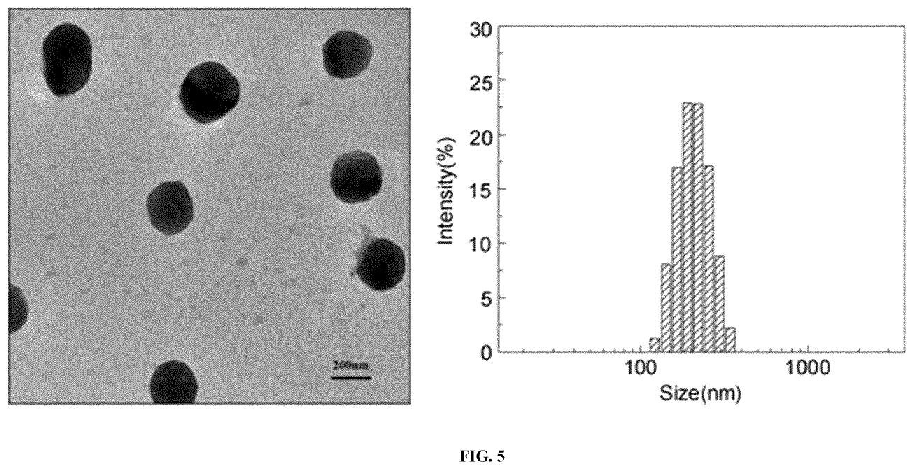

The EVs extracted from the culture supernatant of MCF-7 cells were characterized by TEM images. As shown on the left side of FIG. 5, EVs were in uniformly-dispersed spherical shapes, with a diameter of about 200 nm; the particle size distribution map was shown on the right side of FIG. 5, and the EVs had a particle size distribution of about 211.6 nm, proving that the extracted EVs had a uniform particle size with desirable dispersion.

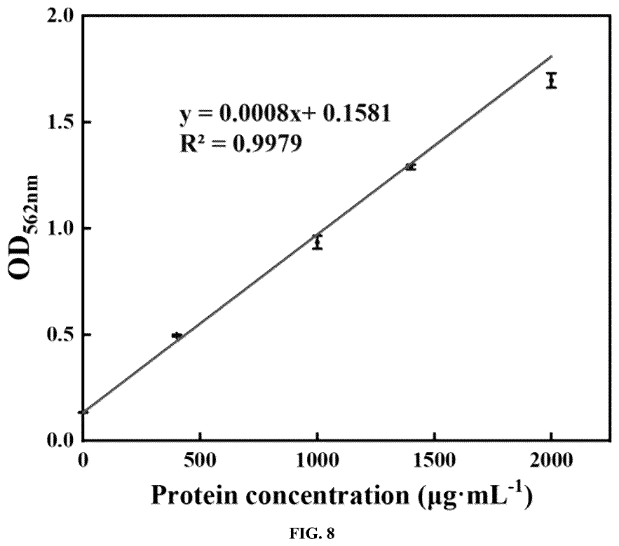

In order to quantify the EVs extracted in each batch, a BCA protein quantification method was used to quantify the protein on EVs, so as to realize standard quantification of the EVs, such that subsequent experiments could standardize the usage of EVs. The absorbance was measured separately at 562 nm for solutions with 5 different protein concentrations (0, 400 μg/mL, 1000 μg/mL, 1400 μg/mL, and 2000 μg/mL), and a standard regression line (FIG. 8) was drawn, to obtain a relationship equation between the protein concentration and absorbance: y=0.0008x+0.1581, R²=0.9979. In subsequent experiments, the absorbance of each batch of newly-extracted EVs was measured, and the corresponding protein content was obtained, which corresponded to the amount of EVs.

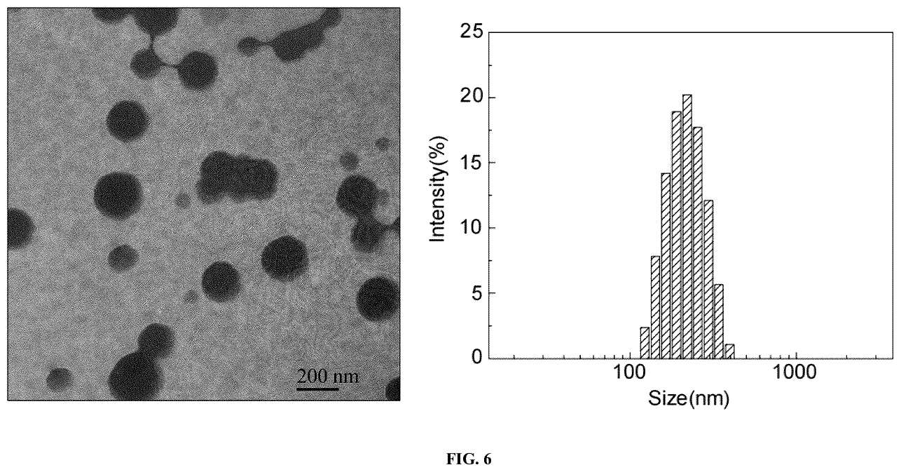

In addition, in order to obtain RVs with a particle size of about 200 nm, repeated extrusion with filters of corresponding specifications was conducted. The particle size of the final RVs could be verified by TEM images (left figure in FIG. 6) as expected. The particle size distribution map (right figure in FIG. 6) showed that the particle size of RVs was uniformly distributed at around 222 nm, once again proving that RVs with uniform particle size were successfully prepared.

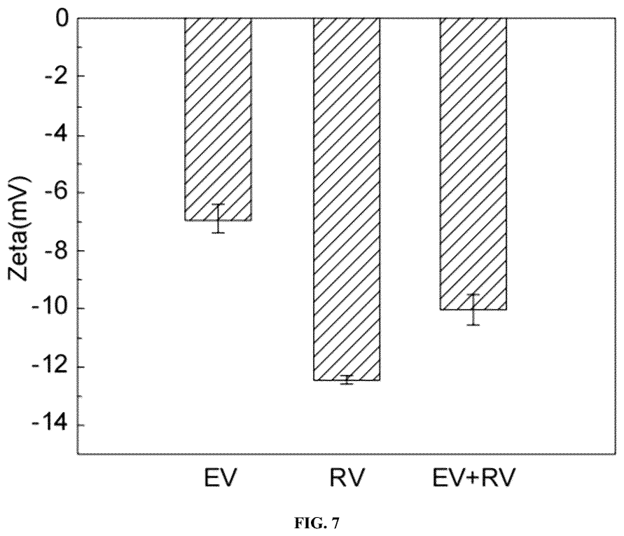

EVs and RVs were incubated at a room temperature for 2 h, and a zeta potential before and after thereof was measured to verify whether membrane fusion between EVs and RVs occurred. The results were shown in FIG. 7. Before incubation, the zeta potentials of EVs and RVs were −6.923 mV and −12.43 mV, respectively; after incubation, the potential became −9.99 mV, and the membrane potential was between the two values before incubation, indicating that the membrane fusion occurred after incubation of EVs and RVs.

### 2.4 In Vitro Fluorescence Detection Ability of DNA Self-Assembly

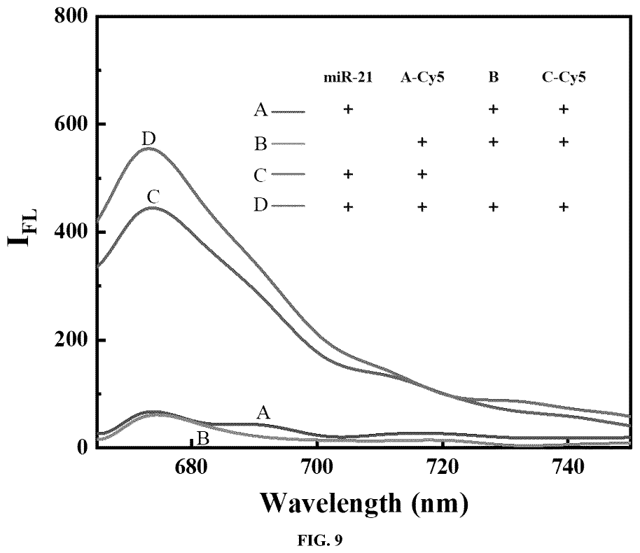

Four sets of control fluorescence detection experiments were designed to evaluate the signal amplification ability of DNA self-assembly using three probes for fluorescence detection of miRNA-21. As shown in FIG. 9, for a blank group (red) without target miRNA-21 and an experimental group without A-Cy5 (black), no obvious fluorescence changes were observed. This indicated that no signaling based on DNA self-assembly occurred, which was consistent with the previous electrophoretic analysis. In addition, the fluorescence signal generated by the presence of all three probes (green) was significantly higher than that of A-Cy5 alone (blue). This proved that the fluorescence intensities of probe A-Cy5 and probe C-Cy5 were superimposed during the self-assembly, and this signal amplification improved the detection sensitivity.

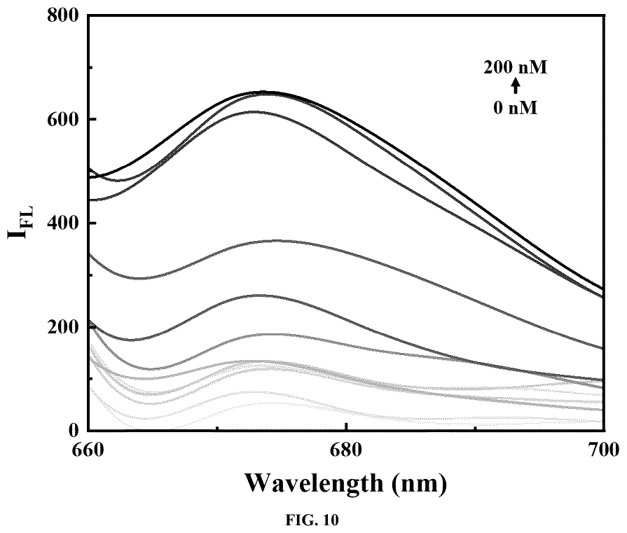

The ability of the DNA self-assembly system to quantitatively detect miRNA-21 in solution was shown in FIG. 10. The fluorescence spectrum from bottom to top in the figure was a fluorescence spectrum curve when the concentration of miRNA-21 increased from 0 nM to 200 nM during the DNA self-assembly. It was seen that as the concentration of the target miRNA-21 increased, the fluorescence intensity of the fluorescence curve at 672 nm also increased.

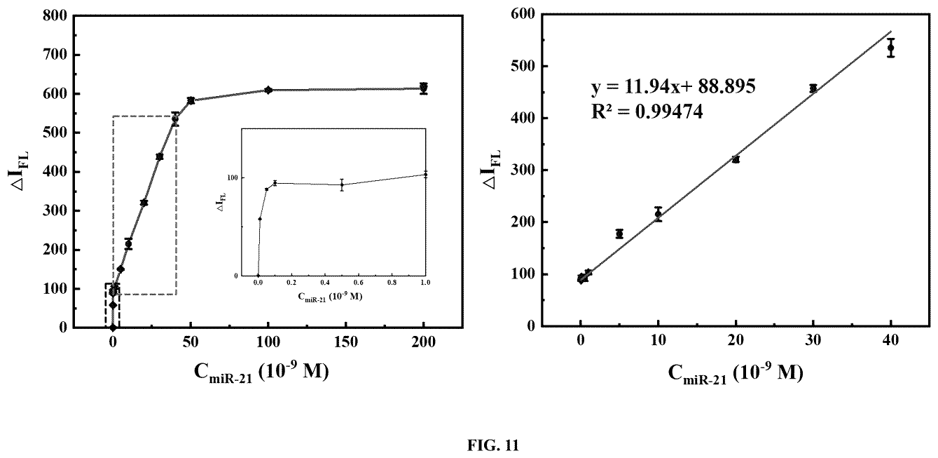

The fluorescence intensities at 672 nm of the solutions obtained by reactions between different concentrations of miRNA-21 and the three probes with a fixed concentration were recorded and plotted in FIG. 11 (left), where the inset was enlarged from a black dotted box. This trend line could more intuitively display a relationship between the concentration of the target detection substance and the fluorescence intensity; the figure on the right was a standard regression line plotted for the data in the red dotted box. As shown in the figure, there was a desirable linear relationship between the fluorescence intensity and the miRNA-21 concentration at 50 pM to 40 nM, and a linear equation was y=11.94x+88.895; where x represented the concentration of miRNA-21, y was a fluorescence intensity change value ΔI_{FL}. (ΔI_{FL}=I_{FLt}−I_{FL0}). These fluorescence measurements demonstrated a high sensitivity of the DNA self-assembly system.

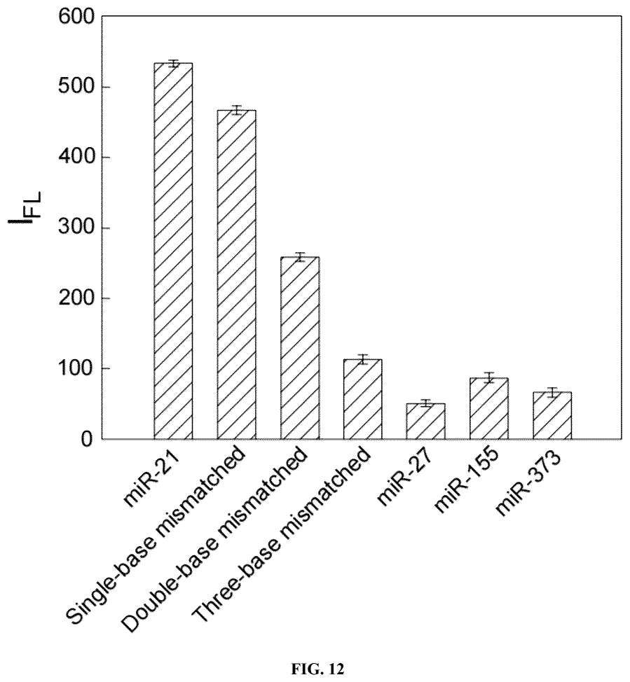

In order to explore the specificity of the DNA self-assembly triggered by the three hairpin probes designed for miRNA-21 to other different types of detection miRNAs, six different miRNAs and miRNA-21 were selected to react with the three probes and fluorescence intensity of each group was detected (FIG. 12). As shown in the figure, the six control groups were a single-base mismatched sequence of miRNA-21, a double-base mismatched sequence of miRNA-21, a three-base mismatched sequence of miRNA-21, miR-27, miR-155, and miR-373. Of these probes tested against miRNA-21, only miRNA-21 efficiently triggered the response and showed strong fluorescence; while all other miRNAs fluoresced far short of this intensity. It was proved that the DNA self-assembly of the three hairpin probes designed by the present disclosure had an extremely high specificity for the target detection substance.

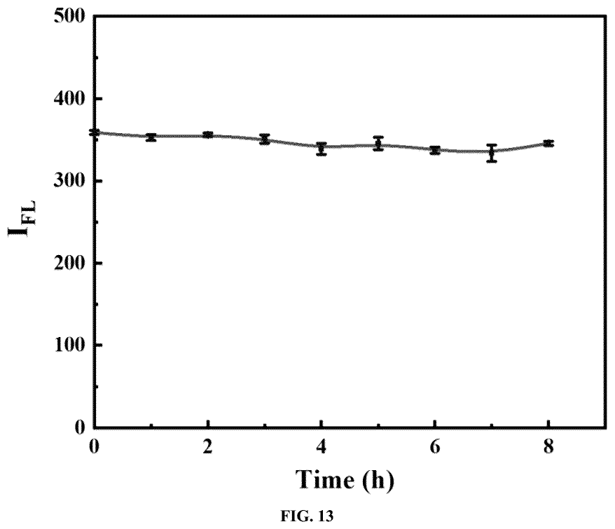

Meanwhile, a stability of the fluorescence signal at 4° C. of a product formed after the DNA self-assembly was also detected. The results were shown in FIG. 13. There was almost no significant change in the fluorescence intensity measured from a moment the self-assembly ended (0 h) to an 8th h, indicating that the DNA self-assembly product had an excellent stability at 4° C.

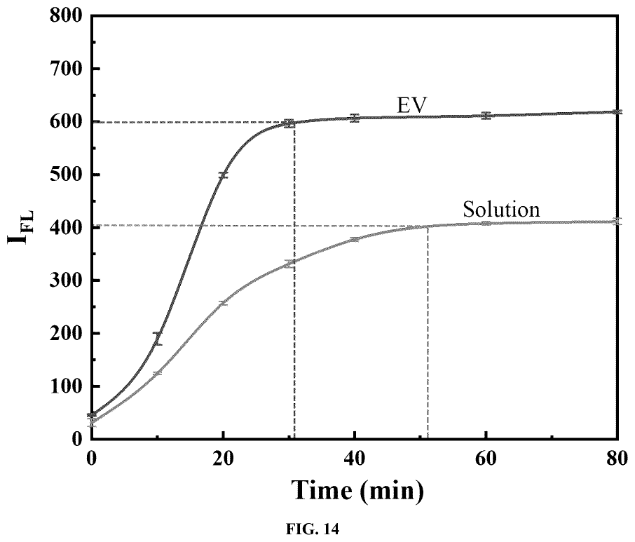

In order to verify that the fluorescence reaction occurring in the confined space of EVs is more efficient than that occurring in solution, the fluorescence intensities of miRNA-21 in EVs and miRNA-21 in solution were further detected with the same concentration of three probes, and changes were recorded in the fluorescence intensity of the two groups within 80 min (FIG. 14). It was clearly seen in the figure that the fluorescence value of the solution group (red) reached a maximum at about 50 min and tended to equilibrated. However, the reaction of the EVs group (black) reached equilibrium in about 30 min and the fluorescence value tended to be stable. Most importantly, the fluorescence intensity of the EVs group increased by about ⅓ compared with that of the solution group, indicating that the efficiency of the DNA self-assembly was improved. The reason was that when the reacting DNA was confined in the nanoscale confined space of EVs, the probability of collision between the probes and the detection target was increased, such that the reaction kinetics become stronger; moreover, the fluorescence signal detected by DNA self-assembly was amplified in the confined space of EVs.

Although the present disclosure has been described in detail through the above examples, the examples are only a part rather than all of the examples of the present disclosure. All other examples obtained by persons based on these examples without creative efforts shall fall within a protection scope of the present disclosure.

1. A method for in situ detection of a micro ribonucleic acid (miRNA) carried by extracellular vesicles (EVs), comprising: subjecting red blood cell membrane (RBCM)-derived vesicles (RVs) to membrane fusion with EVs, and delivering a specific hairpin probe designed for a target miRNA into the EVs to complete DNA self-assembly, thereby achieving in situ fluorescence detection of the miRNA carried by the EVs.

2-14. (canceled)

15. The method according to claim 1, wherein there are three specific hairpin probes; sequences of the three specific hairpin probes each comprise a self-complementary sequence and a complementary palindromic sequence; and two of the specific hairpin probes each are modified with a fluorophore and a quencher.

16. The method according to claim 1, wherein the target miRNA comprises miRNA-21.

17. The method according to claim 16, wherein a specific hairpin probe designed for the miRNA-21 comprises A-Cy5, B, and C-Cy5; A has a nucleotide sequence shown in SEQ ID NO: 1, and the sequence shown in SEQ ID NO: 1 has a quencher modified on T at a 11th position and a fluorophore modified on T at a 53rd position from a 5′-end to a 3′-end;
B has a nucleotide sequence shown in SEQ ID NO: 2; and
C has a nucleotide sequence shown in SEQ ID NO: 3, and the sequence shown in SEQ ID NO: 3 has a fluorophore modified on T at a 6th position and a quencher modified on T at a 54rd position from a 5′-end to a 3′-end.

18. The method according to claim 17, wherein the quencher comprises BHQ2.

19. A system for in situ detection of a miRNA-21 carried by EVs, comprising a miRNA-21 standard and a specific hairpin probe, wherein the specific hairpin probe comprises A-Cy5, B, and C-Cy5; A has a nucleotide sequence shown in SEQ ID NO: 1, and the sequence shown in SEQ ID NO: 1 has BHQ2 modified on T at a 11th position and Cy5 modified on T at a 53rd position from a 5′-end to a 3′-end;
B has a nucleotide sequence shown in SEQ ID NO: 2; and
C has a nucleotide sequence shown in SEQ ID NO: 3, and the sequence shown in SEQ ID NO: 3 has Cy5 modified on T at a 6th position and BHQ2 modified on T at a 54rd position from a 5′-end to a 3′-end.

20. The system according to claim 19, wherein the miRNA-21 standard has a sequence shown in SEQ ID NO: 4.

21. The system according to claim 19, wherein the miRNA-21 standard is diluted with a PBS into different working concentrations during use.

22. The system according to claim 20, wherein the miRNA-21 standard is diluted with a PBS into different working concentrations during use.

23. A kit for in situ detection of a miRNA-21 carried by EVs, comprising the system according to claim 19, an RV extraction reagent, and an EVs extraction reagent.

24. The kit according to claim 23, wherein the miRNA-21 standard has a sequence shown in SEQ ID NO: 4.

25. The kit according to claim 23, wherein the miRNA-21 standard is diluted with a PBS into different working concentrations during use.

26. The kit according to claim 24, wherein the miRNA-21 standard is diluted with a PBS into different working concentrations during use.

27. The kit according to claim 23, wherein the RV extraction reagent comprises a reagent required for preparation of the RVs by extrusion.

28. The kit according to claim 24, wherein the RV extraction reagent comprises a reagent required for preparation of the RVs by extrusion.

29. The kit according to claim 25, wherein the RV extraction reagent comprises a reagent required for preparation of the RVs by extrusion.

30. The kit according to claim 26, wherein the RV extraction reagent comprises a reagent required for preparation of the RVs by extrusion.

31. The kit according to claim 23, wherein the RVs have a particle size of 200 nm.

32. The kit according to claim 24, wherein the RVs have a particle size of 200 nm.

33. The kit according to claim 25, wherein the RVs have a particle size of 200 nm.
